(19) United States
(12) Patent Application Publication
(10) Pub. No.: US 2008/0249482 A1
(43) Pub. Date: Oct. 9, 2008

(54) Self catheterization kit

(76) Inventor: Miki Erez, Miami, FL (US)

Correspondence Address: MARK TERRY, 950 Peninsula Corporate Circle, Suite 3020, Boca Raton, FL, 33487, US

(21) Appl. No.: 11/784,056

(22) Filed: Apr. 5, 2007

(51) Int. Cl. A61M 5/32

(52) U.S. Cl. 604/265

(57) ABSTRACT

A kit for self-catheterization is disclosed. The kit includes a sealed enclosure including a fastener for fastening the enclosure to an individual and self-catheterization supplies located within the enclosure, wherein the supplies include a catheter, lubrication, gloves and an antimicrobial agent. The kit further includes a separate sealable compartment within the enclosure for receiving drained urine during self-catheterization. In one alternative, the fastener comprises a cord for placement around the individual's neck, wherein the enclosure is coupled to both ends of the cord. In another alternative, the fastener comprises a hook and loop element for attaching the enclosure to the individual's clothing.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### FIELD OF THE INVENTION

The invention disclosed broadly relates to the field of catheters, and more particularly relates to the field of self-catheterization kits.

### BACKGROUND OF THE INVENTION

Intermittent catheterization is the temporary placement of a catheter (tube) to remove urine from the body. This is usually done by placing the catheter through the urethra (the tube that leads from the bladder to the outside opening) to empty the bladder. People of all ages may require catheterization for a short period of time or on an occasional basis. Intermittent (short-term) catheterization may be necessary for people with neurological disorders, women who have undergone certain gynecological surgeries, and anyone who is unable to properly empty the bladder. The goal of intermittent catheterization, typically required every 3 hours, is to prevent urinary tract infections, further bladder or kidney damage, and to completely empty the bladder. Most people are able to learn how to perform this procedure themselves, i.e., self-catheterization.

To perform clean intermittent self-catheterization (CISC), the individual must learn the basic location of the important components in the urinary system. Additionally, the person must have the physical ability to reach the urethra, and must be able to move the equipment as necessary. People who are unable to see the urethra may be taught how to feel for the proper location of the urethral opening. Catheters that are used for CISC are slightly different from the catheters used for long-term catheterization. A long-term catheter is held in place by a balloon—CISC catheters do not have this balloon or the side port, found at the end of the catheter, for balloon inflation. CISC catheters may be made of a clear plastic or a softer rubber material. These catheters also come in a variety of sizes. CISC catheters are intended to be disposable.

Performing CISC generally consists of the following steps. (Note that only CISC for males is described below.) First, all CISC equipment is assembled. This can include the catheter, lubricant, drainage receptacle, rubber gloves, disinfectant, etc. Next, the individual washes his hands thoroughly with soap and water and cleans the penis and urethral opening. Note that the individual's hands must be kept completely disinfected throughout this process. The individual then places sterile medical gloves on his hands. Then, the catheter is lubricated and the penis is held perpendicular to the body. Subsequently, the catheter is inserted into the urethra and advanced into the bladder. Next, the flow of urine begins and the catheter is held in place until the urine flow stops and the bladder is empty. Then, the catheter is withdrawn slowly to make sure the entire bladder empties. Since the catheter is disposable, it is discarded. Additionally, the drainage receptacle holding the evacuated urine, the medical gloves, lubricant container and all other supplies used during the self-catheterization process must be discarded.

As the description of CISC above shows, the self-catheterization process typically requires a generally sterile setting that includes a sterile, flat surface such as a counter or table, easy access to a sterile water faucet and water basin and surroundings that are free from germs and other bacteria that may contaminate the items used during CISC. The difficulty of performing the CISC process above is further compounded when the individual performing the process is sitting in a wheelchair.

Any harmful microbe that contaminates the CISC process may end up within the individual's bladder and cause an infection. Thus, the individuals performing CISC must be extremely careful with the choice of location for performing CISC. Unfortunately, individuals do not always have the option of choosing an ideal location for performing CISC. Individuals may find themselves at a football stadium, a wedding, on travel or at a festival where the only option for performing CISC is a public bathroom stall, a small, non-sterile place or out in the open. In these cases, it is very difficult for individuals to perform CISC, since their only choice is a generally dirty setting that lacks a clean flat surface, provides poor access to a sterile water faucet and basin and the surroundings are ripe with germs and other bacteria. As such, due to their affliction, these individuals are often resigned to staying at home or within reach of a location with the proper facilities for CISC.

Therefore, a need exists to overcome the problems with the prior art as discussed above, and particularly for a more efficient way to facilitate self-catheterization for individuals in locations lacking the proper facilities.

### SUMMARY OF THE INVENTION

Briefly, according to an embodiment of the present invention, a kit for self-catheterization is disclosed. The kit includes a sealed enclosure including a fastener for fastening the enclosure to an individual and self-catheterization supplies located within the enclosure, wherein the supplies include a catheter, lubrication, gloves and an antimicrobial agent. The kit further includes a separate sealable compartment within the enclosure for receiving drained urine during self-catheterization. In one alternative, the fastener comprises a cord for placement around the individual's neck, wherein the enclosure is coupled to both ends of the cord. In another alternative, the fastener comprises a hook and loop element for attaching the enclosure to the individual's clothing.

In another embodiment of the present invention, a self-catheterization kit is disclosed. The self-catheterization kit includes self-catheterization supplies including a catheter, lubrication, gloves and an antimicrobial agent and a sealed enclosure defining a sterile inner volume for holding the self-catheterization supplies. The self-catheterization kit further includes a cord for placement around an individual's neck, wherein the enclosure is coupled to both ends of the cord, and a separate sealable compartment within the enclosure for receiving drained urine during self-catheterization.

In another embodiment of the present invention, a self-catheterization kit is disclosed. The self-catheterization kit includes a sealed enclosure defining a sterile inner volume and at least one compartment within the inner volume of the enclosure for holding a catheter, lubrication, gloves and an antimicrobial agent. The self-catheterization kit further includes a fastener for fastening the enclosure to an individual, wherein the fastener is coupled to an outside surface of the enclosure, and a sealable compartment within the enclosure for receiving drained urine during self-catheterization.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings. Additionally, the left-most digit of a reference number identifies the drawing in which the reference number first appears.

FIG. 1 is an illustration of a perspective view of a self-catheterization kit, according to one embodiment of the present invention.

FIG. 2 is an illustration of a perspective view of the self-catheterization kit of FIG. 1 in the open position.

FIG. 3A is an illustration of a frontal view of the self-catheterization kit of FIG. 1 as worn on a standing male individual, according to one embodiment of the present invention.

FIG. 3B is an illustration of a frontal view of the self-catheterization kit of FIG. 1 as worn on a standing female individual, according to one embodiment of the present invention.

FIG. 4A is an illustration of a frontal view of the self-catheterization kit of FIG. 1 as worn on a male individual in a wheelchair, according to one embodiment of the present invention.

FIG. 4B is an illustration of a frontal view of the self-catheterization kit of FIG. 1 as worn on a female individual in a wheelchair, according to one embodiment of the present invention.

FIG. 5 is an illustration of a medical glove kit, according to one embodiment of the present invention.

### DETAILED DESCRIPTION

The present invention comprises a completely self-contained self-catheterization kit comprised in a wearable pack. The self-catheterization kit provides users who must perform self-catheterization intermittently throughout the day with a kit that facilitates carrying and use in any place and at any time. The self-catheterization kit can be used in non-sterile areas and/or areas that have no surfaces on which to place items. The self-catheterization kit can further be used in small, confined areas, such as in a public bathroom stall. As such, the self-catheterization kit can also be used as an emergency self-catheterization kit. The self-catheterization kit of the present invention can be used anywhere, even when a proper facility is available.

The self-catheterization kit of the present invention is self-contained. That is, all items necessary for self-catheterization are included in the self-catheterization kit. Use of the self-catheterization kit eliminates the need for outside support, such as running water or a counter on which to place items. The self-catheterization kit includes at least a catheter, lubrication for inserting the catheter into the urethra, hand gloves and an antimicrobial agent such as iodine (in the form of towelettes, cotton balls or wipes) for disinfecting the hands, the urethra, or the penis/vagina before and/or after self-catheterization. The self-catheterization kit may also include a receptacle for receiving the evacuated urine. The present invention eliminates the need for running water, a sterile, flat surface, a sterile water basin and can be used anywhere. The self-contained nature of the invention is beneficial since users of self-catheterization cannot rely on the availability of tools necessary for the self-catheterization process in public places or at others' residences.

The self-catheterization kit of the present invention is further wearable such that it can be worn on the user's body. For example, the kit may be suspended from a cord or necklace that is worn around the user's neck, attached to an article of clothing such as a vest, a jacket or the frontal thigh area of the pants, or integrated into an article of clothing, such as a vest, a jacket, a shirt or a pair of pants. This feature allows the user to easily access all items within the kit without touching other, non-sterile, objects. This aspect of the invention is beneficial since users of self-catheterization cannot rely on the sterility of objects around them (surfaces, door handles, faucet valves, etc.) during the self-catheterization process. This aspect of the invention is further beneficial for individuals that use crutches or arm supports or individuals that necessitate the use of wheelchairs, since they possess a limited range of movement.

In one alternative, the self-catheterization kit of the present invention arranges the self-catheterization supplies within the kit separately, so as not touch each other. That is, all supply items necessary for self-catheterization are included in small, self-contained compartments or enclosures within the self-catheterization kit. This eliminates the spread of germs or bacteria from one tool to another during or after the self-catheterization process. This feature of the present invention is beneficial since users of self-catheterization must maintain full sterility of their hands, their urethra and all self-catheterization tools during the self-catheterization process.

In one alternative, after use, the self-catheterization kit can be sealed so as to seal in all self-catheterization supplies that were used during the self-catheterization process. This facilitates safe transportation of the self-catheterization kit (no spillage) to a waste receptacle. The urine receptacle in particular is independently sealable so as to allow the user to carry the used receptacle and the used, sealed self-catheterization kit without spillage until he can properly discard it.

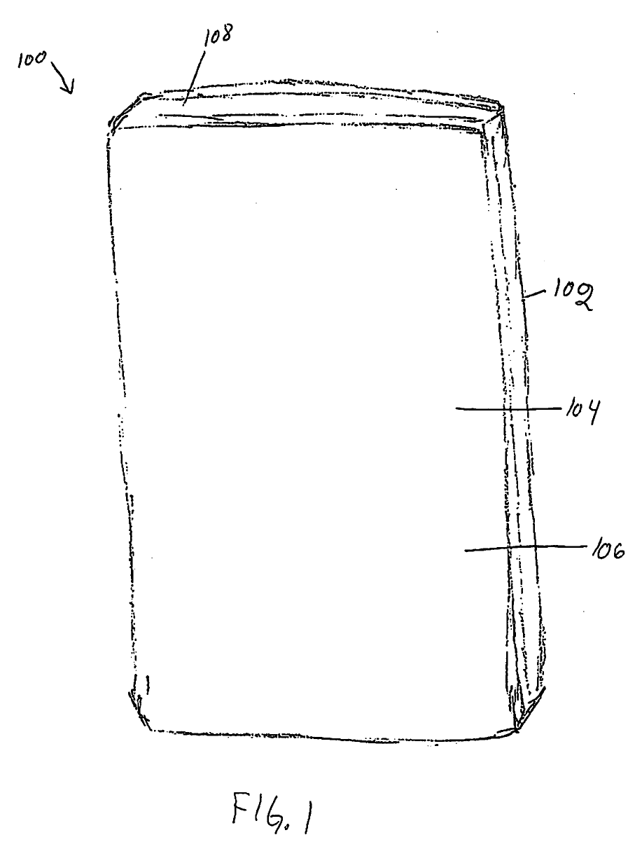

FIG. 1 is an illustration of a perspective view of a self-catheterization kit 100, according to one embodiment of the present invention. Self-catheterization kit 100 comprises a sealed enclosure 102 defining a sterile inner volume 104 for holding a variety of self-catheterization supplies for performing CISC, such as a catheter, lubrication, hand gloves and an antimicrobial agent. In one embodiment of the present invention, the enclosure 102 comprises a planar plastic sack having a removable face 106, described in greater detail below. Plastic covers a range of synthetic or semisynthetic polymerization products. Plastics are composed of organic condensation or addition polymers and may contain other substances to improve performance or economics. In the present invention, plastic may comprise any one of the following forms of plastic: polyethylene, polystyrene, high impact polystyrene, polyethylene terephthalate and polyvinylidene chloride.

In one embodiment of the present invention, the enclosure 102 includes a fastener for fastening the enclosure to an individual. The fastener may comprise a cord for placement around the individual's neck, wherein the enclosure 102 is coupled to both ends of the cord. In this embodiment, a tear-away section 108 of the enclosure 102 exposes the cord when the section 108 is removed. Alternatively, the fastener may comprise a hook and loop element for attaching the enclosure to the individual's clothing.

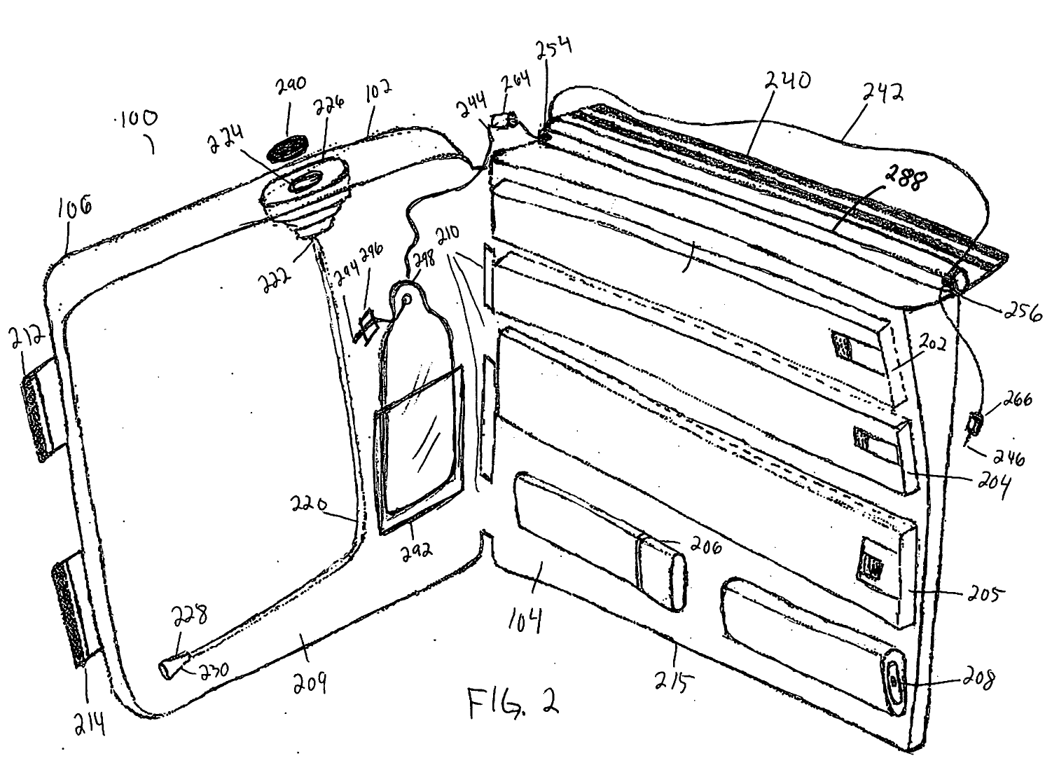

FIG. 2 is an illustration of a perspective view of the self-catheterization kit 100 of FIG. 1 in the open position. The enclosure 102 of the self-catheterization kit 100 comprises a planar plastic sack having a removable face 106. Hook and loop tabs 212, 214 removably couple removable face 106 to panel 215 of the enclosure 102. When hook and loop tabs 212, 214 are disconnected from panel 215, the removable face 106 is opened via hinge or seam 210 (or removed altogether), and the sterile inner volume 104 of the enclosure 102 is exposed.

The enclosure 102 of the self-catheterization kit 100 comprises a variety of separate and sterile compartments 202, 204, 205, 206, 208 and 209 for holding a variety of self-catheterization supplies. Compartments 202, 204 may hold a cover-all 212 which may comprise a sterile parcel of fabric or cotton-based paper that is placed over the crotch-area of the individual during CISC so as to protect the CISC supplies from being contaminated by other portions of the individual's body, the individual's clothes or other portions of the individual's genitalia. the cover-all 212 may include a hole or orifice through which the individual's penis may be inserted so to allow access to the penis but protect the CISC supplies from being contaminated by other areas.

Compartments 206, 208 may hold an antimicrobial agent such as iodine (in the form of towelettes, cotton balls or wipes) for disinfecting the hands, the urethra, or the penis/vagina before and/or after self-catheterization. Disinfectants are antimicrobial agents that are applied to non-living objects to destroy microorganisms, the process of which is known as disinfection. Sanitizers are high level disinfectants that kill over 99.9% of a target microorganism in applicable situations. Some disinfectants offer complete sterilization, while remaining inexpensive, and non-corrosive. Some disinfectants have a wide spectrum (kill nearly all microorganisms), whilst others kill a smaller range of disease-causing organisms but are preferred for other properties (they may be non-corrosive, non-toxic, or inexpensive). Other possible types of disinfectants include alcohol-based and hydrogen peroxide-based substances.

Compartments 206, 208 may also hold a catheter lubricant, which is a specialized lubricants which serves to reduce friction within the urethra or other body parts. These lubricants provide lubrication during the CISC process. Water-based personal lubricants are water soluble and are generally the type that is least irritating to body surfaces (particularly mucous membranes). Silicone-based lubricants are oils, as they are chemically hydrophobic. They tend to retain lubrication longer than water-based lubricant.

Compartment 205 may hold medical gloves, which are medical safety accessories that ensure sanitary hospital conditions by limiting the individual's exposure to infectious matter. Medical gloves also serve to protect the individual from disease through contact with bodily fluids. Medical gloves are traditionally made of latex and powdered with cornstarch. Due to the increasing rate of latex allergy among health professionals as well as in the general population, there has been an increasing move to medical gloves made of non-latex materials such as vinyl or nitrile rubber.

Panel 106 of the enclosure 102 includes a sealable compartment 209 that includes a catheter 220. A catheter is a tube that can be inserted into a body cavity duct or vessel. Catheters thereby allow drainage or injection of fluids or access by surgical instruments. The process of inserting a catheter is catheterization. In most uses a catheter is a thin, flexible tube, or a “soft” catheter. In some uses, a catheter is a larger, solid tube, or a “hard” catheter. Placement of a catheter into a particular part of the body may allow draining urine from the urinary bladder as in urinary catheterization, when the urethra is damaged as in suprapubic catheterization or if the urethra is too narrow to allow normal urination. A catheter may also allow drainage of fluid collections, such as an abdominal abscess, administration of intravenous fluids, medication or parenteral nutrition, angioplasty, angiography, balloon septostomy, balloon sinuplasty. Additionally, a catheter may allow direct measurement of blood pressure in an artery or vein or direct measurement of intracranial pressure. Finally, a catheter may allow administration of anaesthetic medication into the epidural space, the subarachnoid space, or around a major nerve bundle such as the brachial plexus.

The catheter 220 is located within the compartment 209 and the working end 222 of the catheter 220 may be pulled out of the orifice 224, around which a semi-rigid collar 226 is placed. The other end 228 of the catheter 220 includes a flaring element 230 with a diameter that is larger than the diameter of orifice 224 so as to prevent the entire catheter 220 from being removed from the compartment 209. This arrangement allows for the catheter 220 to be pulled out of the orifice 224 to allow for insertion of the end 222 into the individual's urethra. During draining, the urine evacuated from the individual's bladder is drained into the sealable compartment 209. After draining, the catheter 220 is placed back into the compartment 209 via orifice 224. A cap 290 may be placed on orifice 224 so as to prevent spillage or leakage of the urine from sealable compartment 209 after use.

In one embodiment of the present invention, a separate sealable compartment may be present behind panel 215. The sealable compartment may be accessible via a sealable seam 288 located at the top of panel 215. This sealable compartment may be used to hold discarded items used during the self-catheterization process. Because the compartment is sealable, it can be used to safely hold and transport the used items until they can be discarded safely in an appropriate place. In another embodiment of the present invention, the separate sealable compartment discussed above is the compartment 209.

FIG. 2 further shows a mirror 298 for placement into a pocket 292 located on panel 106. The mirror 298 may be a non-breakable mirror for use by females, overweight males or any individual who cannot see his/her urethra during the self-catheterization process. The mirror 298 is used to aid in seeing the urethra and facilitating insertion of the catheter into the genitalia. The mirror 298 is attached to the enclosure 102 via a cord 294, wherein the cord 294 includes a draw tab 296 that allows the tightening of the cord 294 as it is threaded through the draw tab and fixed in position.

FIG. 2 further shows a tear-away section 240 located along the top seam, which may be rigid, of the enclosure 102. When the section 240 is removed, a cord 242 is exposed along the top seam of the enclosure 102. The cord 242 is for placement around the individual's neck during use. Each end 244, 246 of the cord 242 is threaded through an orifice 254, 256, respectively, in the enclosure 102. Each end 244, 246 of the cord 242 includes a draw tab 264, 266, respectively, wherein each draw tab 264, 266 allows the tightening of the cord 242 as the cord 242 is threaded through the draw tab and fixed in position.

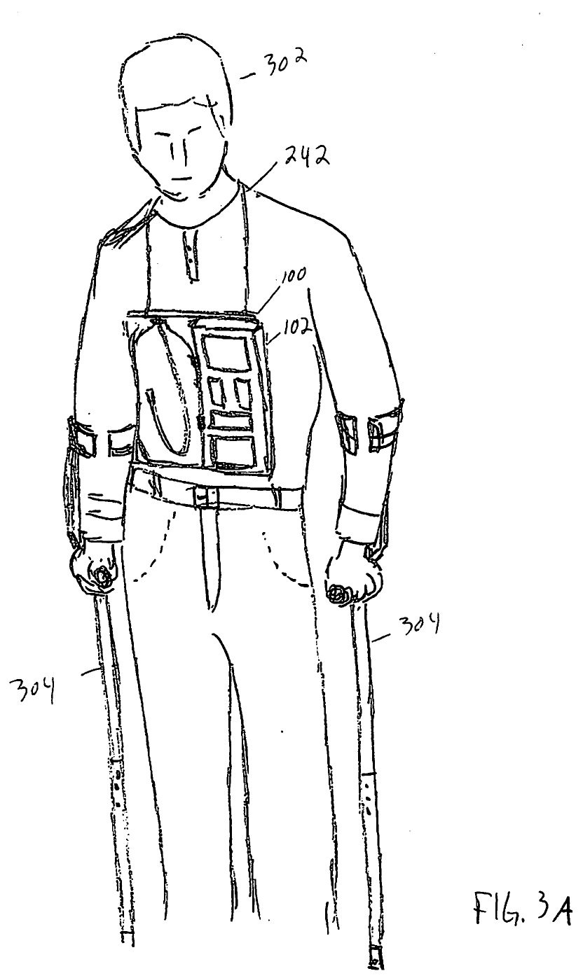

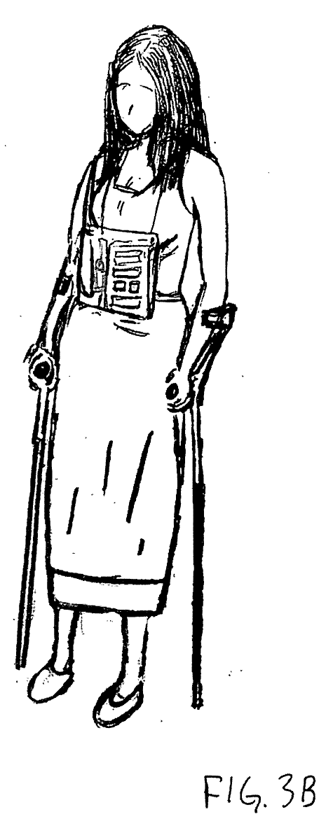

FIG. 3A is an illustration of a frontal view of the self-catheterization kit 100 of FIG. 1 as worn on a standing male individual 302, according to one embodiment of the present invention. FIG. 3A shows that the individual 302 necessitates the use of crutches or arm supports 304 to facilitate standing or walking. FIG. 3A shows that the cord 242 attached to the enclosure 102 of the self-catheterization kit 100 is placed around the neck of the individual 302 such that the self-catheterization kit 100 hangs at about chest-level on the individual 302. This allows the individual 302 easy access to the items within the enclosure 102 so as to facilitate the performance of the CISC procedure while standing up. FIG. 3B is an illustration of a frontal view of the self-catheterization kit 100 of FIG. 1 as worn on a standing female individual, according to one embodiment of the present invention.

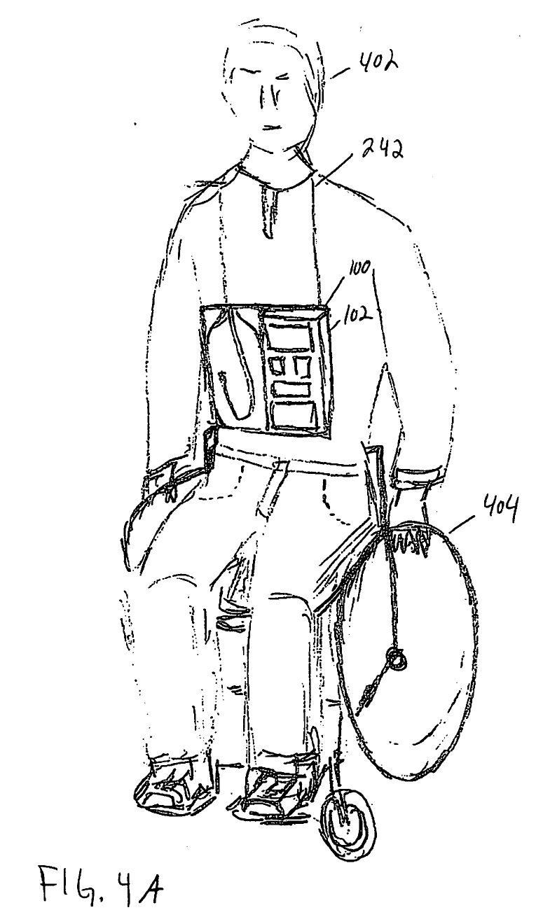

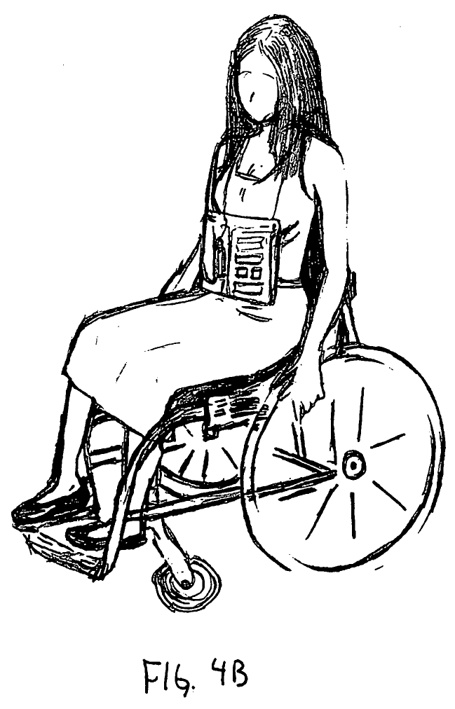

FIG. 4A is an illustration of a frontal view of the self-catheterization kit 100 of FIG. 1 as worn on a male individual 402 in a wheelchair 404, according to one embodiment of the present invention. FIG. 4A shows that the individual 402 necessitates the use of a wheelchair 404. FIG. 4A shows that the cord 242 attached to the enclosure 102 of the self-catheterization kit 100 is placed around the neck of the individual 402 such that the self-catheterization kit 100 hangs at about chest-level on the individual 402. This allows the individual 402 easy access to the items within the enclosure 102 so as to facilitate the performance of the CISC procedure while sitting in the wheelchair 404. FIG. 4B is an illustration of a frontal view of the self-catheterization kit 100 of FIG. 1 as worn on a female individual in a wheelchair, according to one embodiment of the present invention.

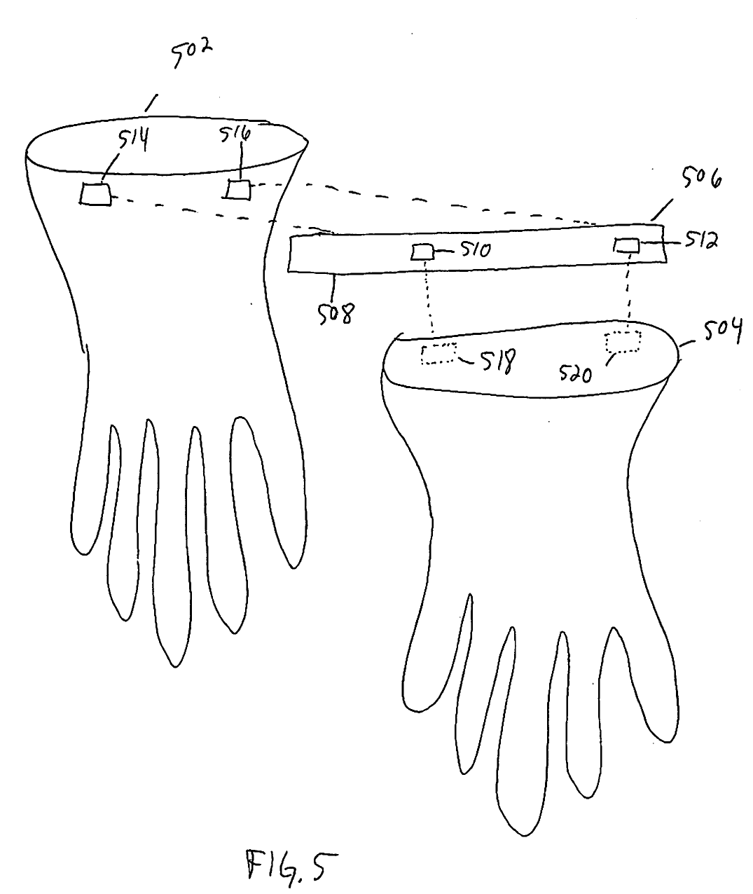

As described above, compartment 205 of FIG. 2 may hold medical gloves. FIG. 5 is an illustration of a medical glove kit 500, according to one embodiment of the present invention. Medical glove kit 500 shows a first medical glove 502 and a second medical glove 504. Also shown is a rigid strip 506 comprising a rigid, elongated planar element. One end 508 of the rigid strip 506 is used by the individual to hold the rigid strip 506 with his hand. The rigid strip 506 includes two portions 510, 512 of one side of a hook and loop fastener on one side of the rigid strip 506. The rigid strip 506 further includes two portions of one side of a hook and loop fastener on the other side (not shown) of the rigid strip 506.

The first medical glove 502 includes two portions 514, 516 of one side of a hook and loop fastener at the base of the first medical glove 502. Portions 514, 516 removably connect to two portions of one side of a hook and loop fastener on the other side (not shown) of the rigid strip 506. The second medical glove 504 includes two portions 518, 520 (partially occluded) of one side of a hook and loop fastener at the base of the second medical glove 504. Portions 518, 520 removably connect to two portions 510, 512 of one side of a hook and loop fastener on the rigid strip 506.

Thus, in order to place the gloves 502, 504 on the individual's hands, the individual may hold end 508 of the rigid strip 506 with a first hand, insert a second hand into the second glove 504, and remove the second glove 504 from the rigid strip 506 by detaching portions 518, 520 from portions 510, 512. The individual may then hold end 508 of the rigid strip 506 with the second hand, insert the first hand into the first glove 502, and remove the first glove from the rigid strip 506 by detaching portions 514, 516 from hook and loop portions on the rigid strip 506.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

We claim:

1. A kit, comprising:
a sealed enclosure including a fastener for fastening the enclosure to an individual;
self-catheterization supplies located within the enclosure, wherein the supplies include a catheter, lubrication, gloves and an antimicrobial agent; and
a separate sealable compartment within the enclosure for receiving drained urine during self-catheterization.

2. The kit of claim 1, wherein the fastener comprises a cord for placement around the individual's neck, wherein the enclosure is coupled to both ends of the cord.

3. The kit of claim 2, wherein the enclosure comprises a planar plastic sack having a removable face.

4. The kit of claim 3, wherein the enclosure comprises a separate compartment that holds at least one of the self-catheterization supplies before use.

5. The kit of claim 3, wherein the self-catheterization supplies include a glove kit, comprising:
a rigid strip including at least one fastener;
a first glove, wherein the at least one fastener is removably connected to a base of the first glove; and
a second glove, wherein the at least one fastener is removably connected to a base of the second glove,
wherein the individual may hold the rigid strip with a first hand, insert a second hand into the second glove, remove the second glove from the rigid strip, hold the rigid strip with the second hand, insert the first hand into the first glove, and remove the first glove from the rigid strip.

6. The kit of claim 1, wherein the fastener comprises a hook and loop element for attaching the enclosure to the individual's clothing.

7. The kit of claim 6, wherein the enclosure comprises a separate compartment that holds at least one of the self-catheterization supplies before use.

8. The kit of claim 7, wherein the enclosure comprises a separate sealable compartment for holding the self-catheterization supplies after use.

9. The kit of claim 8, wherein the self-catheterization supplies include a glove kit, comprising:
a rigid strip including a hook and loop fastener on each side of the strip;
a first glove, wherein the fastener is removably connected to a base of the first glove; and
a second glove, wherein the fastener is removably connected to a base of the second glove,
wherein the individual may hold the rigid strip with a first hand, insert a second hand into the second glove, remove the second glove from the rigid strip, hold the rigid strip with the second hand, insert the first hand into the first glove, and remove the first glove from the rigid strip.

10. A self-catheterization kit, comprising:
self-catheterization supplies including a catheter, lubrication, gloves and an antimicrobial agent;
a sealed enclosure defining a sterile inner volume for holding the self-catheterization supplies;
a cord for placement around an individual's neck, wherein the enclosure is coupled to both ends of the cord; and
a separate sealable compartment within the enclosure for receiving drained urine during self-catheterization.

11. The self-catheterization kit of claim 10, wherein the enclosure comprises a planar plastic sack having a removable face.

12. The self-catheterization kit of claim 11, wherein the enclosure comprises a separate compartment that holds at least one of the self-catheterization supplies before use.

13. The self-catheterization kit of claim 12, wherein the enclosure comprises a separate sealable compartment that holds the self-catheterization supplies after use.

14. The self-catheterization kit of claim 13, wherein the self-catheterization supplies include a glove kit, comprising:
a rigid strip including at least one fastener;
a first glove, wherein the at least one fastener is removably connected to a base of the first glove; and
a second glove, wherein the at least one fastener is removably connected to a base of the second glove,
wherein the individual may hold the rigid strip with a first hand, insert a second hand into the second glove, remove the second glove from the rigid strip, hold the rigid strip with the second hand, insert the first hand into the first glove, and remove the first glove from the rigid strip.

15. A self-catheterization kit, comprising:
a sealed enclosure defining a sterile inner volume;
at least one compartment within the inner volume of the enclosure for holding a catheter, lubrication, gloves and an antimicrobial agent;
a fastener for fastening the enclosure to an individual, wherein the fastener is coupled to an outside surface of the enclosure; and
a separate sealable compartment within the enclosure for receiving drained urine during self-catheterization.

16. The self-catheterization kit of claim 15, wherein the fastener comprises a cord for placement around the individual's neck, wherein the enclosure is coupled to both ends of the cord.

17. The self-catheterization kit of claim 18, wherein the enclosure comprises a planar plastic sack having a removable face.

18. The self-catheterization kit of claim 17, wherein the enclosure comprises a separate compartment that holds at least one of the self-catheterization supplies before use.

19. The self-catheterization kit of claim 18, wherein the enclosure comprises a separate sealable compartment that holds the self-catheterization supplies after use.

20. The self-catheterization kit of claim 18, further comprising a glove kit, including:
a rigid strip including at least one fastener;
a first glove, wherein the at least one fastener is removably connected to a base of the first glove; and
a second glove, wherein the at least one fastener is removably connected to a base of the second glove,
wherein the individual may hold the rigid strip with a first hand, insert a second hand into the second glove, remove the second glove from the rigid strip, hold the rigid strip with the second hand, insert the first hand into the first glove, and remove the first glove from the rigid strip.
